(19) United States
(12) Patent Application Publication
(10) Pub. No.: US 2026/0199163 A1
(43) Pub. Date: Jul. 16, 2026

(54) AUTOMATED DYNAMIC PATIENT POSITIONING SYSTEM FOR ROBOTIC SURGERY

(71) Applicant: Automated Intelligent Robotics LLC, Sarasota, FL (US)

(72) Inventors: Thomas Medrick Sweeney II, Myakka City, FL (US); Michael Todd Boyer, Venice, FL (US); John Douglas Kuczynski, Sarasota, FL (US); Thomas Medrick Sweeney, III, San Diego, CA (US); Daniel Davenport, Collegeville, PA (US); Vincent E. Mandes, Warrington, PA (US)

(73) Assignee: Automated Intelligent Robotics LLC, Sarasota, FL (US)

(21) Appl. No.: 19/374,668

(22) Filed: Oct. 30, 2025

Related U.S. Application Data
(60) Provisional application No. 63/833,179, filed on Nov. 1, 2024.
(60) Provisional application No. 63/871,709, filed on Aug. 28, 2025.

(51) Int. Cl. A61G 13/06; A61G 13/04; A61G 13/12

(52) U.S. Cl. CPC A61G 13/06; A61G 13/04; A61G 13/1295; A61G 2200/325

(57) ABSTRACT

A dynamic patient positioning system for robotic surgery includes a base for supporting a patient, multiple motion platforms for engaging the patient, a navigation system with fiducial markers for tracking patient position, and a controller coordinating system operation. The motion platforms may be six degrees of freedom hexapods or Stewart platforms that can automatically adjust patient positioning in real-time. The navigation system continuously tracks fiducial marker positions, and the controller automatically repositions the motion platforms when patient movement is detected, maintaining precise positioning throughout surgical procedures. The system provides enhanced surgical accuracy, reduced procedure time, and improved patient outcomes in robotic surgical applications.

### CROSS REFERENCE TO RELATED APPLICATIONS

The present application claims the benefit of U.S. Provisional Application No. 63/833,179 entitled “AUTOMATED DYNAMIC PATIENT POSITIONER FOR USE IN ROBOTIC SURGERY,” and U.S. Provisional Ser. No. 63/871,709 entitled “SEGMENTAL REGISTRATION FOR BONE TRACKING,” the entire disclosures of which are hereby incorporated by reference in their entirety for all purposes.

### BACKGROUND

The present disclosure relates to patient positioning systems for surgical procedures, and more particularly to automated dynamic patient positioning systems that use motion platforms and real-time tracking to maintain and adjust patient positioning.

Robotic surgery has become increasingly prevalent in modern medical practice, offering enhanced precision, reduced invasiveness, and improved patient outcomes. However, a significant challenge in robotic surgery is maintaining consistent patient positioning throughout the procedure. Patients may move due to breathing, muscle relaxation under anesthesia, or external forces applied during surgery. Even small movements can compromise the accuracy of robotic surgical systems, potentially leading to suboptimal outcomes or the need to re-register the patient's position.

Current patient positioning systems are primarily static, consisting of articulating arms, pads, straps, and sometimes motorized actuators. While some dynamic positioning systems exist, such as articulated surgical tables, these systems typically require manual adjustment and do not automatically compensate for patient movement during surgery. As such, there still exists a need for a patient positioning system that can work in conjunction with robotic surgical systems to continuously monitor and maintain proper patient positioning throughout surgical procedures.

### BRIEF SUMMARY OF THE INVENTION

The following presents a simplified summary in order to provide a basic understanding of some aspects of one or more exemplary embodiments or examples of the present teachings. This summary is not an extensive overview, nor is it intended to identify key or critical elements of the present teachings, nor to delineate the scope of the disclosure. Rather, its primary purpose is merely to present one or more concepts in simplified form as a prelude to the detailed description presented later. Additional goals and advantages will become more evident in the description of the figures, the detailed description of the disclosure, and the claims.

The foregoing and/or other aspects and utilities embodied in the present disclosure may be achieved by providing an automated dynamic patient positioning system for robotic surgery. The system includes a base for supporting a patient, a plurality of motion platforms for operatively engaging the patient, a navigation system for tracking the position of the patient including a fiducial marker for attachment to the patient, and a controller operatively in communication with the plurality of motion platforms and the navigation system.

According to aspects illustrated herein, each of the plurality of motion platforms may be a six degrees of freedom motion platform, such as a Stewart platform or hexapod-type robot. The motion platforms may include positioning arms connected to the base, and may further include static bases connected to the positioning arms, dynamic platforms for operatively engaging the patient, and actuators connecting the static bases to the dynamic platforms for movement in three-dimensional space.

Another aspect of the exemplary embodiments is that the system may include compression layers extending from the motion platforms for interfacing with the patient, which may be implemented as beanbag layers or other conformable materials. The navigation system may be an optical or electromagnetic navigation system, and may include navigation sensors for detecting the position of fiducial markers. In some examples, the patient positioning system may include a sensor operatively in communication with the controller for detecting an attribute of the patient. The sensor may be a pressure sensor operatively in communication with the controller for detecting applied forces on the patient and providing feedback to the controller for detecting patient movement.

According to yet other aspects of the exemplary embodiments, the controller may include memory having computer instructions executable to register a predetermined position of the patient, track the position of the fiducial marker in real time, determine if the patient has moved from the predetermined position, and automatically adjust the position of the motion platforms to realign the patient when movement is detected.

According to other aspects described herein, a patient positioning system for surgical procedures includes a base for supporting a patient, a plurality of motion platforms for operatively engaging the patient, a navigation system for tracking a position of the patient including a fiducial marker for attachment to the patient, and a controller. Each of the plurality of motion platforms may be a six degrees of freedom motion platform. The controller is operatively in communication with the plurality of motion platforms and the navigation system. The controller includes a memory having computer instructions executable to register a predetermined position of the patient based on a position of the plurality of motion platforms and the fiducial marker, track the position of the fiducial marker in real time via the navigation system, determine if the position of the patient has moved from the predetermined position based on the tracked position of the fiducial marker, and adjust the position of at least one of the plurality of motion platforms to realign the patient to the predetermined position when it is determined that the patient has moved from the predetermined position.

Exemplary embodiments are described herein. It is envisioned, however, that any system that incorporates features of apparatus and systems described herein are encompassed by the scope and spirit of the exemplary embodiments. Therefore, the foregoing general description of the illustrative embodiments and the following detailed description thereof are merely exemplary aspects of the teachings of this disclosure and are not restrictive.

### BRIEF DESCRIPTION OF THE DRAWINGS

The foregoing summary, as well as the following detailed description of the exemplary embodiments of the subject disclosure, will be better understood when read in conjunction with the appended drawings. For the purpose of illustrating the subject disclosure, there are shown in the drawings exemplary embodiments. It should be understood, however, that the exemplary embodiments are not limited to the precise arrangements and instrumentalities shown. In the drawings:

FIG. 1 is a perspective view of a patient positioning system in accordance with an exemplary embodiment of the subject disclosure;

FIG. 2 is a partial perspective view of the patient positioning system of FIG. 1;

FIG. 3 is a rear elevational view partially in section of the patient positioning system of FIG. 1;

FIG. 4 is a front elevational view partially in section of the patient positioning system of FIG. 1;

FIG. 5 is a perspective view of an exemplary sensor applicable to the patient positioning system in accordance with another exemplary embodiment of the subject disclosure; and

FIG. 6 is a schematic block diagram of a controller applicable to the exemplary embodiments of the patient positioning system of the subject disclosure.

### DETAILED DESCRIPTION

Reference will now be made in detail to the various exemplary embodiments of the subject disclosure illustrated in the accompanying drawings. Wherever possible, the same or like reference numbers will be used throughout the drawings to refer to the same or like features. It should be noted that the drawings are in simplified form and are not drawn to precise scale.

We initially point out that description of well-known starting materials, processing techniques, components, equipment and other well-known details may merely be summarized or are omitted so as not to unnecessarily obscure the details of the present disclosure. Thus, where details are otherwise well known, we leave it to the application of the present disclosure to suggest or dictate choices relating to those details. The drawings depict various examples related to embodiments of illustrative methods, apparatuses, and systems for automated dynamic patient positioning.

Certain terminology is used in the following description for convenience only and is not limiting. Directional terms such as top, bottom, left, right, above, below and diagonal, are used with respect to the accompanying drawings. The term “distal” shall mean away from the center of a body. The term “proximal” shall mean closer towards the center of a body and/or away from the “distal” end. The words “inwardly” and “outwardly” refer to directions toward and away from, respectively, the geometric center of the identified element and designated parts thereof. Such directional terms used in conjunction with the following description of the drawings should not be construed to limit the scope of the subject disclosure in any manner not explicitly set forth. Additionally, the term “a,” as used in the specification, means “at least one.” The terminology includes the words above specifically mentioned, derivatives thereof, and words of similar import.

“About” as used herein when referring to a measurable value such as an amount, a temporal duration, and the like, is meant to encompass variations of ±20%, ±10%, ±5%, ±1%, or ±0.1% from the specified value, as such variations are appropriate.

“Substantially” as used herein shall mean considerable in extent, largely but not wholly that which is specified, or an appropriate variation therefrom as is acceptable within the field of art. “Exemplary” as used herein shall mean serving as an example.

Throughout this disclosure, various aspects of the subject disclosure can be presented in a range format. It should be understood that the description in range format is merely for convenience and brevity and should not be construed as an inflexible limitation on the scope of the subject disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The term “controller” or “control system” is used herein generally to describe various apparatus such as a computing device relating to the operation of one or more device that directs or regulates a process or machine. A controller can be implemented in numerous ways (e.g., such as with dedicated hardware) to perform various functions discussed herein. A “processor” is one example of a controller which employs one or more microprocessors that may be programmed using software (e.g., microcode) to perform various functions discussed herein. A controller may be implemented with or without employing a processor, and may be implemented as a combination of dedicated hardware to perform some functions and a processor (e.g., one or more programmed microprocessors and associated circuitry) to perform other functions. Examples of controller components that may be employed in various embodiments of the present disclosure include, but are not limited to, conventional microprocessors, application specific integrated circuits (ASICs), and field-programmable gate arrays (FPGAs).

Exemplary embodiments as disclosed herein may also include computer-readable media for carrying or having computer-executable instructions or data structures stored thereon. Such computer readable media can be any available media that can be accessed by a general purpose or special purpose computer. By way of example, and not limitation, such computer-readable media can comprise RAM, ROM, EEPROM, CD-ROM or other optical disk storage, magnetic disk storage or other magnetic storage devices, or any other medium which can be used to carry or store desired program code means in the form of computer-executable instructions or data structures.

When information is transferred or provided over a network or another communications connection (either hardwired, wireless, or combination thereof) to a computer, the computer properly views the connection as a computer-readable medium. Thus, any such connection is properly termed a computer-readable medium. Combinations of the above should also be included within the scope of the computer-readable media.

Computer-executable instructions include, for example, instructions and data which cause a general-purpose computer, special purpose computer, or special purpose processing device to perform a certain function or group of functions. Computer-executable instructions also include program modules that are executed by computers in stand-alone or network environments. Generally, program modules include routines, programs, objects, components, and data structures, and the like that perform particular tasks or implement particular abstract data types. Computer-executable instructions, associated data structures, and program modules represent examples of the program code means for executing steps of the methods disclosed herein. The particular sequence of such executable instructions or associated data structures represents examples of corresponding acts for implementing the functions described therein.

Although the exemplary embodiments of the subject disclosure are not limited in this regard, discussions utilizing terms such as, for example, “processing,” “tracking,” “computing,” “calculating,” “determining,” “using,” “establishing”, “analyzing”, “checking”, or the like, may refer to operation(s) and/or process(es) of a controller, computer, computing platform, computing system, or other electronic computing device, that manipulate and/or transform data represented as physical (e.g., electronic) quantities within the computer's registers and/or memories into other data similarly represented as physical quantities within the computer's registers and/or memories or other information storage medium that may store instructions to perform operations and/or processes.

Furthermore, the described features, advantages and characteristics of the exemplary embodiments of the subject disclosure may be combined in any suitable manner in one or more embodiments. One skilled in the relevant art will recognize, in light of the description herein, that the present disclosure can be practiced without one or more of the specific features or advantages of a particular exemplary embodiment. In other instances, additional features and advantages may be recognized in certain embodiments that may not be present in all exemplary embodiments of the subject disclosure.

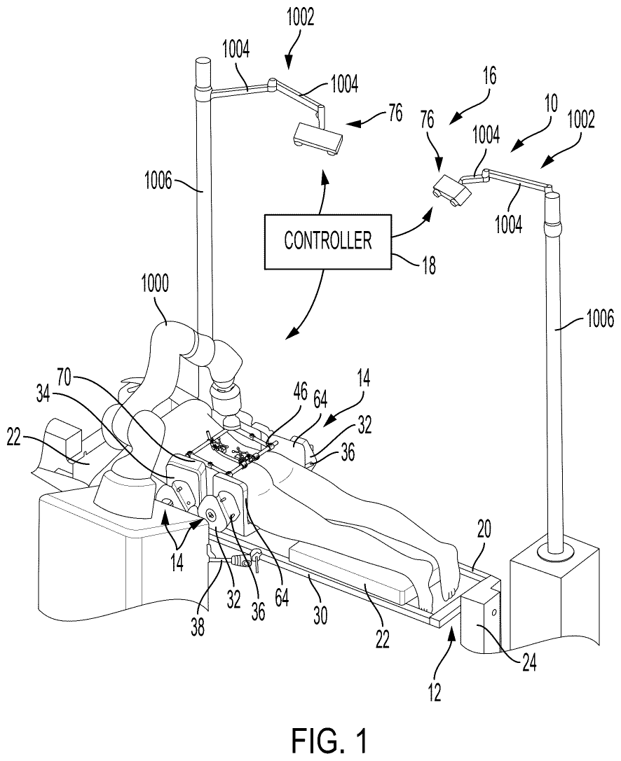

FIG. 1 depicts an exemplary embodiment of a patient positioning system 10 for surgical procedures. The patient positioning system 10 includes a base 12 for supporting a patient, motion platforms 14, a navigation system 16, and a controller 18 The patient positioning system may be used in robotic surgical procedures with e.g., a robot 1000 that is distinct and separate from the patient positioning system but which may be operatively connected to or in communication with so as to operate cooperatively therewith.

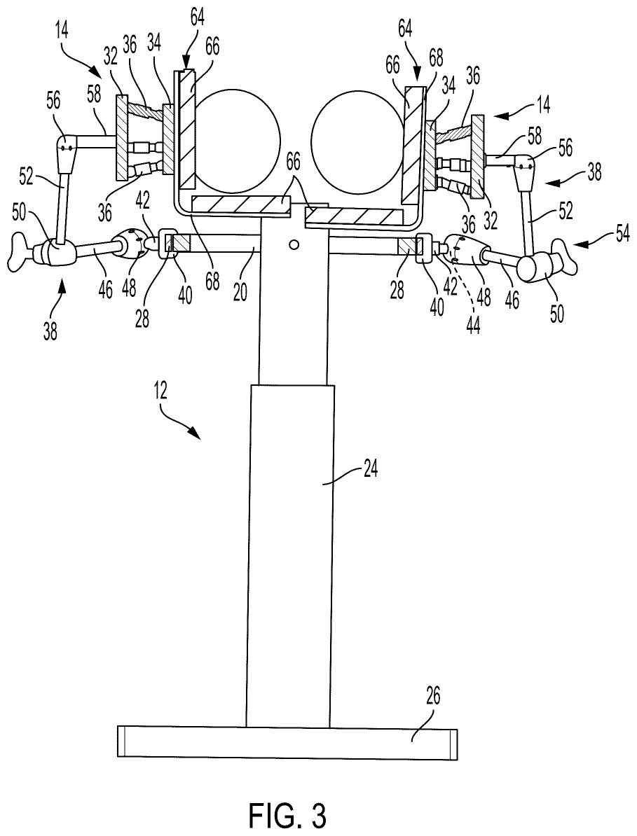

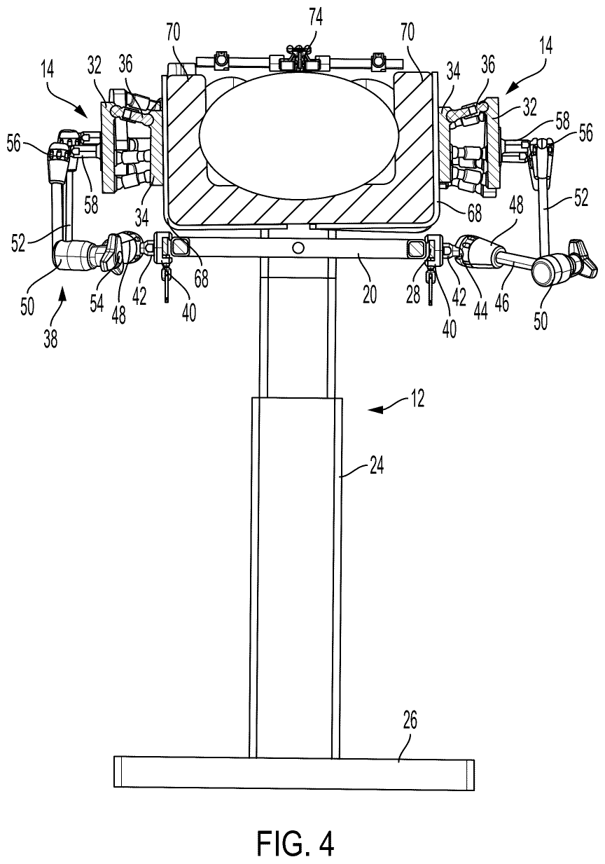

The base 12 serves as a surgical table for the patient positioning system 10 and is configured to support the patient during surgical procedures, as well understood by a skilled artisan. The base 12 may be designed to accommodate the patient in various surgical positions, including e.g., a prone position for spinal procedures, etc. The base 12 may include a superior frame 20, patient platforms 22, legs 24 and a frame base 26 (FIGS. 3-4). While not being limited to a particular structure, the superior frame 70 may have an open frame modular design with rigid lateral sides 28 ideal for supporting interchangeable patient platforms 22 that may sit on or otherwise attach to the superior frame and support a patient resting thereon. The patient platforms 22 may be pads, mattresses, or combination of such with imaging plates and the like.

The superior frame 20 may attach to the frame base 26 via one or more legs 24 that descend from the superior frame downwards to the frame base. The legs 24 may be adjustable (e.g., telescopic) for height adjustments. The frame base 26 may be a rigid platform that spans the length of the superior frame 20 and provides stability, mobility and powered articulation to the adjustable legs and superior frame. For example, the frame base 26 may include casters (not shown) that roll on a floor for mobility and pivot joints connecting the frame base with the legs 24 to enable rotation and articulation of the superior frame 20 top.

The frame base 26 may also include a motor (not shown) for providing powered articulation of the base 12 and additional approaches to adjust patient position. For example, each leg 24 of the base 12 may include an individual motor and each motor may be operatively in communication with the controller 18 for operatively controlling patient position.

The motion platforms 14 e.g., a plurality of motion platforms, may be connected to various mounting points of the base 12, such as along the lateral sides of the base, to operatively engage and/or support various parts of the patient positioned on the base. The motion platforms 14 provide controlled movement capabilities for patient positioning during surgical procedures. In an exemplary embodiment, the motion platforms 14 include at least two motion platforms positioned about each lateral side 30 of the base 24. Alternatively, the motion platforms 14 can include two, three, four, or five motion platforms along each lateral side 30 of the base. The motion platforms 14 may be positioned in pairs for engagement at opposing sides of a patient's body region to allow for bilateral positioning control and coordinated movement and support of different body parts. The motion platforms include a static platform 32, a dynamic platform 34, and actuators 36 that connect the static platform 32 to the dynamic platform for movement of the dynamic platform in three-dimensional space relative to the static platform.

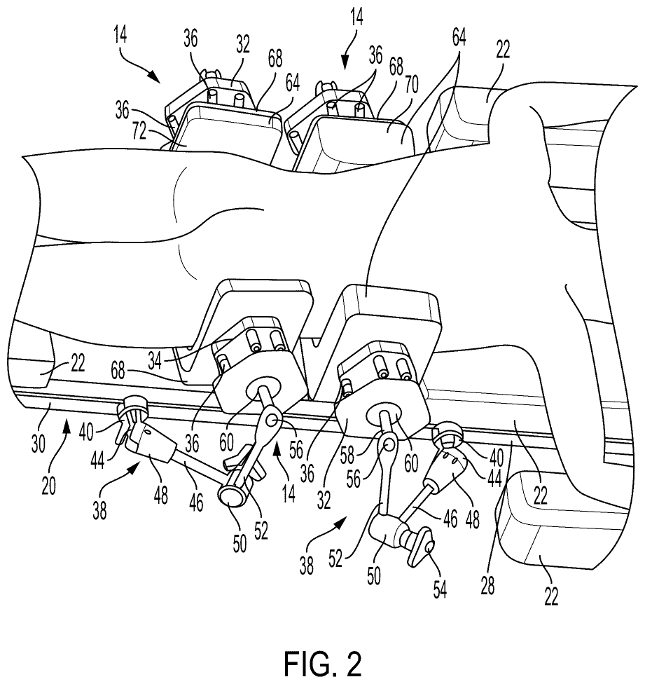

Referring to FIGS. 2-4, each of the motion platforms 14 may be configured as a six degree of freedom motion platform, also referred to as a hexapod. Exemplary hexapods applicable to the patient position system of the subject disclosure include the Stewart platform by Axiom Optics (see https://www.axiomoptics.com/products/hexapods/), the disclosure of which is hereby incorporated by reference in its entirety for all purposes.

The static platform 32 that can be weight bearing having a surface that provides a fixed reference base for other motion platform components. Static platforms 32 are not limited to any particular shape and size, and may be shaped in a general planar configuration, for example, as a rigid board with the reference base surface on a first side, and a thickness/material to maintain structural integrity under dynamic loads. The static platform 32 are connected to the base 12 via connectors that enable coupling of the static platform 32 to the base 12 and to other aspects of the motion platform, as will be discussed in greater detail below.

The static platform 32 may be connected to the base 12 directly or via structural linkage, such as a positioning arm 38, that may be connected to both the base and the static platform. The positioning arm 38 may be used as an intermediary interface having adjustable and fixed support between the base 12 and the static platform 32.

In accordance with an exemplary embodiment, the positioning arm 38 includes mechanical linkages for attachment to the base. For example, referring to FIGS. 1-3, each positioning arm 38 includes a clamp 40 rigidly connected to the lateral side 30 of the base 12 with a rod 42 extending from a free end of the clamp to a distal end e.g., a spherical end 44. The spherical end 44 may be the ball joint of a ball and socket joint. The positioning arm 38 also includes an arm rod 46 having a female socket 48 a one end designed to receive and hold the spherical end 44 to allow for rotation and articulation between the clamp and the arm rod.

The arm rod 46 extends from the socket 48 to a mechanical elbow joint 50 that allows angular movement between the arm rod and a forearm rod 52 of the positioning arm 38. One of the arm rod and forearm rod may include a swivel bolt 54 about the elbow joint 50 that extends through an aperture of each rod. In examples, the swivel bolt 54 may have a threaded distal end for threaded engagement with a locking nut that may be rotated on the swivel bolt to compress the joint and allow or prevent the angular movement between the arm rod and forearm rod. The forearm rod 52 may extend to a distal wrist joint 56, such as a ball and socket joint that allows rotation and articulation between the forearm rod and a clamp rod 58 connected to the motion platform, with one of the forearm rod and clamp rod having a ball joint and the other rod having a socket joint.

The clamp rod 58 extends from the ball and socket joint 56 and is coupled to the static platform 32 via a rigid joint 60. The positioning arm 38 depicted in the figures is merely one example of an intermediary interface that may extend from a connection about a lateral side 30 of the base 12 and may position the motion platform 14as desired adjacent to a patient body part for support and adjustment thereof, and other examples are considered within the scope of the subject disclosure.

As noted above, each motion platform 14 further includes a dynamic platform 34 for operatively engaging the patient, and actuators 36 that connect the static platform 32 to the dynamic platform for movement of the dynamic platform in six degrees of freedom relative to the static platform. Like the static platform 32, the dynamic platform 34 may be a rigid board with a thickness/material that maintains structural integrity under dynamic loads. The dynamic platforms are operational to move in relation to the static platform 32 in response to actuator 36 movement. The dynamic platforms 34 may include a patient interface surface or coupling interface for patient contact that contacts and accommodates different anatomical regions as needed to position, adjust and hold the patient.

As shown by example in FIGS. 2-4, the actuators 36 may connect the static platform 32 to the dynamic platform 34 via spherical or universal joints 62 for controlled movement of the dynamic platform. The actuators 36 may be configured as linear actuators or servo-controlled struts that extend and retract along their axis to generate the coordinated movements that result in the desired positioning of the dynamic platform 34. In some examples, the actuators may be rotary actuators. While not being limited to a particular theory, a plurality of actuators (e.g., at least two, 3-6) may be arranged between the static platform 32 and the dynamic platform 34 in a hexagonal configuration, with each actuator independently controlled to provide six degrees of freedom positioning capability, allowing the dynamic platform to move in translation along three orthogonal axes and rotate about three orthogonal axes for precise control over position and orientation of the dynamic platform 34 and associated patient contact surfaces. The actuators 36 may be arranged in a parallel kinematic configuration for high precision, stiffness and load capacity.

The actuators are operatively connected to or in communication with the controller 18 or may be controlled via control signals from the controller. The control signals may be forwarded from the controller 18 via user input. Control signals may also be forwarded automatically from the controller to move the actuators 36 and orient the dynamic platform 34 and associated contact surfaces to an ideal position or to reposition the dynamic platform upon detected patient movement or surgical requirements, as will also be discussed in greater detail below.

Still referring to FIGS. 1-4, in accordance with another exemplary embodiment, the patient positioning system 10 includes a compression layer 64 for interfacing with the patient. The compression layer 64 is positioned adjacent to the motion platforms for being operatively engaged by the motion platforms and serves as an intermediary interface between the motion platforms and the patient. The compression layer 64 may provide controlled contact and force distribution across different anatomical regions of the patient to enhance patient comfort and safety. For example, the compression layer may include different types of patient contact surfaces having various levels of cushioning, elasticity, rigidity and conformance tailored to accommodate various patient anatomical regions. In some examples, the compression layer 64 may include pads 66 that may be rigid for boney areas like the pelvis, to provide firm support and precise positioning control for skeletal structures that can withstand higher contact forces. In other examples, the pads 66 may be more compliant and configured for interaction with soft tissue areas.

FIG. 3 depicts the base 12, motion platforms 14 and compression layer 64 along with a cross-sectional view of a patient's legs. The compression layer 64 may be positioned to support the weight of the patient; that is, may be positioned under the patient. In accordance with an exemplary embodiment, an angle bracket 68 or L-bracket may be secured to the dynamic platform 34 and extend downwardly and under the patient to support the patient as needed. The compression layer 64 may attach to vertical and horizontal sides of the angle bracket 68 with a pad 66 at a lateral side of a patient and a pad 66 under the patient for multi-axial support of the associated anatomical region. In some examples, the pad 66 may be attached to both interior side surfaces of the angle bracket 68. The compression layer pad 66 may be integral across both interior side surfaces.

An exemplary compression layer 64 may include a beanbag 70 operable to conform to irregular anatomical contours and provide distributed support for patient regions (e.g., midsection, soft tissue areas) that may be sensitive to concentrated forces. FIG. 4 is a partial section view of the compression layer 64 with the base 12 and motion platforms 14 depicted in FIGS. 1 and 2. The beanbag 70 may be a soft, flexible support pad filled with beanbag material (e.g., small pellets, beads, memory foam) that allows the bag to mold to the patient's anatomical shape and provide multi-surface support of the associated body part. Referring to FIG. 4, the beanbag 70 is shown attached to the interior and bottom side surfaces of the angle brackets 68. The beanbag 70 may wrap around the side and anterior aspects of the patient, with opposing side motion platforms 14 controlling the position of the beanbag.

In examples, the beanbag 70 may be a vacuum activated beanbag that can transition between flexible and rigid states based on vacuum activation, as understood by a skilled artisan. When vacuum is not applied, the vacuum activated beanbag 70 may remain flexible and conformable, allowing the beanbag material to flow and adapt to patient contours. Upon vacuum activation, the vacuum activated beanbag 70 may become rigid and maintain its shape, effectively holding the engaged patient region (e.g., midsection, long bone, femur) in a desired configuration that matches the patient's anatomical contours.

Another exemplary compression layer applicable to the subject disclosure may comprise polyurethane expanding foam. The polyurethane expanding foam compression layer is configured to conform to the patient's body and then harden over a short time period so as to provide a patient-specific compression layer. Alternative compression layers can include compression layers having viscoelastic gel layers or compression layers having fluidized positioners.

While the exemplary compression layer i.e., beanbag 70 in FIG. 4, is shown as an integral compression layer spanning under and around the patient, it is understood that the compression layer 64 may be a plurality of distinct compression layers, each attached to a different area of the angle brackets 68. For example, the compression layer 64 may be two beanbags separated between the angle brackets 68, with each beanbag attached to a respective angle bracket. In some examples, a greater number of beanbags 70 may be used, with each beanbag attached to a different interior surface of the angle brackets 68. Using multiple beanbags, or segmenting beanbags into a number of compartments, assists to keep pellets or beads within the beanbags spread across the horizontal and vertical interior sides of the angle brackets.

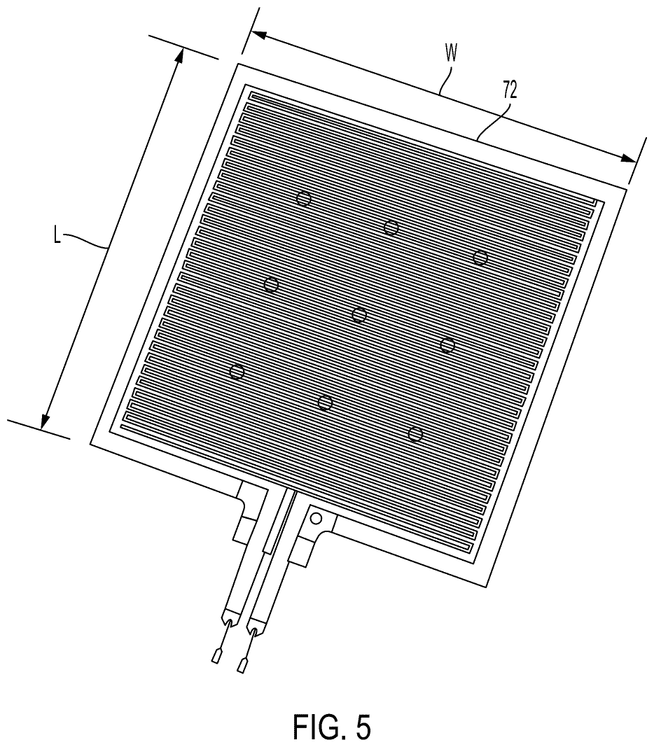

FIG. 5 depicts an exemplary sensor 72 applicable to the patient positioning system in accordance with another exemplary embodiment of the subject disclosure to detect an attribute of the patient. For example, the sensor 72 may be a pressure sensor that detects contact forces between the motion platforms 14 and the patient. As such, the pressure sensor may be incorporated into or behind the patient contacting surface of the compression layer 64. Multiple or a plurality of sensors 72 may be employed at various locations of the compression layer or other patient engaging locations, with each sensor being operatively engaged with or operatively in communication with the controller 18.

According to an aspect, the sensor 72 can have a length “L” of about 10 mm to about 10 inches, about 20 mm to about 5 inches, about 30 mm, 40 mm, 50 mm, 60 mm, 3 inches or 4 inches and a width “W” of about 10 mm to about 10 inches, about 20 mm to about 5 inches, about 30 mm, 40 mm, 50 mm, 60 mm, 3 inches or 4 inches. The sensor 72 may have a thickness substantially less than its width and length, and sufficient to generate electrical signals that correspond to applied forces and allow the patient positioning system 10 to monitor mechanical forces between the motion platforms 14 and the patient. In this manner, the pressure sensor 72 may provide dual benefits within the patient positioning system 10, functioning both as a safety mechanism to prevent excessive or unintentional force application to the patient, and as a feedback system for detecting patient movement.

Referring back to FIG. 1, the navigation system 16 is configured for tracking a position of the patient and instruments used during surgery of the patient. In examples, the navigation system may include a fiducial marker 74 operable for attachment to the patient and a navigation sensor 76 configured to detect the position and orientation of the fiducial marker. While not being limited to a particular theory, the navigation system 16 may include an optical tracking system, an electromagnetic tracking system and/or another tracking system configured to track the position and orientation of fiducial markers 74 attached to the patient or instruments used on the patient. For an optical tracking system, the navigation sensor 76 may include a camera (e.g., infrared (IR)-sensitive, Charge-Coupled Device (CCD), Complementary Metal-Oxide-Semiconductor (CMOS)) that captures the 2D positions of the fiducial marker 74 with line-of-sight visibility. An optical fiducial marker 74 may be active and emit IR light (e.g., LEDs) and/or may be passive and reflect IR light from an external source. With a second navigation sensor camera offset from the first camera, the navigation system 16 via controller 18 may use triangulation to reconstruct the 3D position and orientation of the fiducial marker 74 for real-time tracking of the marker.

The navigation sensor(s) 76 may be positioned to maintain optimal detection conditions throughout the range of patient positioning adjustments that may occur during surgical procedures. As shown in FIG. 1, the sensors 76 may be mounted well above the operating area of the patient where they avoid interference with surgical operations. For example, the sensors 76 may be attached to placement stands 1002 having swing arms 1004 coupled to the sensors and extending to freestanding poles 1006.

For an electromagnetic tracking system, the navigation sensor 76 may include a transmitter that emits a low-frequency electromagnetic field in the tracking space around the patient, and the fiducial marker 74 may include a receiver (e.g., tiny coil embedded therein) that detects the magnetic field and communicates with the controller 18 to process the signals and track the receivers without requiring line-of-sight visibility. It is understood that the navigation system 16 is not limited to optical tracking systems or electromagnetic tracking systems, as other tracking systems may also be used within the scope of the examples. Such other tracking systems may include inertial tracking systems that may use combinations of accelerometers, gyroscopes and magnetometers; ultrasound-based tracking systems that may use ultrasound pulses and time-of-flight measurements; radio frequency (RF) and RFID tracking; and hybrid systems that combine multiple tracking modalities. A hybrid approach may allow the navigation system to maintain tracking continuity when one tracking system may be temporarily compromised by patient position, anatomical obstructions (e.g., line-of-sight) or surgical procedures.

For more optimal tracking capabilities, the navigation system 16 may include a plurality of the fiducial markers 74 that attach to the patient. Exemplary fiducial markers may include pins 78, wires, probes, or other connectors or clamps configured for attachment to a patient body part, as readily understood by a skilled artisan. For example, fiducial markers may be attached to vertebra in the spine to track the position and orientation of a patient's spine. In other examples, fiducial markers may be attached to fractured segments of a bone to track individual segments during fracture fixation.

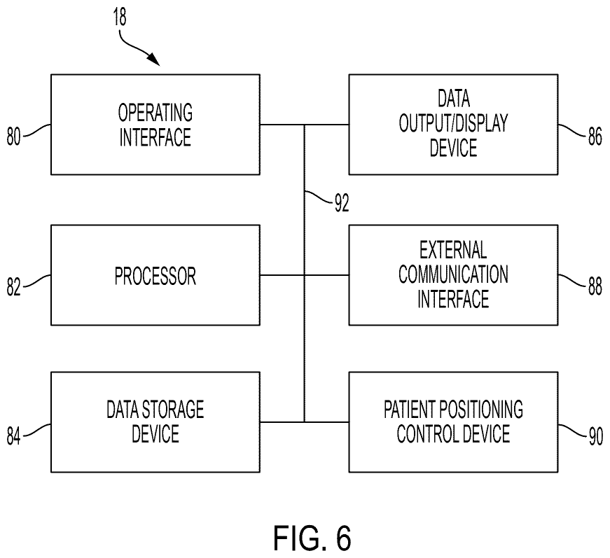

FIG. 6 illustrates a block diagram of the controller 18 for executing instructions to automatically control the patent positioning system 10 and components thereof. The exemplary controller 18 may provide input to or be a component of a controller for executing patient positioning aspects for automatically adjusting patient positioning including patient position registration, tracking, movement and adjustments in a system such as that depicted in FIGS. 1-4, and described in greater detail below.

The controller 18 may include an operating interface 80 by which a user may communicate with the exemplary patient positioning system 10. The operating interface 80 may be a locally-accessible user interface associated with the patient positioning system. The operating interface 80 may be configured as one or more conventional mechanism common to controllers and/or computing devices that may permit a user (e.g., surgeon, medical staff) to input information to the controller 18. The operating interface 80 may include, for example, a conventional keyboard, a touchscreen with “soft” buttons or with various components for use with a compatible stylus, a microphone by which the user may provide oral commands to the controller 18 to be “translated” by a voice recognition program, or other like device by which the user may communicate specific operating instructions to the exemplary controller. The operating interface 80 may be a part or a function of a graphical user interface (GUI) mounted on, integral to, or associated with, the motion platform, navigation system and/or positioning system 10 with which the controller 18 is associated.

The controller 18 may include one or more local processors 82 for individually operating the controller 18 and for carrying into effect control and operating functions for comprehensive monitoring and adjustment of patient position throughout surgical procedures, including coordinating the operation of motion platforms, tracking systems, and patient monitoring components. For example, in real-time upon receipt of patient position information from fiducial markers and sensors, processors may trigger an automated motion platform to engage, manipulate, position, reposition and/or hold bones at an anatomical area of interest (e.g., spine, long bone, pelvic region, torso) and stabilize the patient position at that area. Processor(s) 82 may include at least one conventional processor or microprocessor that interprets and executes instructions to direct specific functioning of the controller 18, and control patient anatomical position with the exemplary controller.

The controller 18 may include one or more data storage devices 84. Such data storage device(s) 84 may be used to store data or operating programs to be used by the controller 18, and specifically the processor(s) 82. Data storage device(s) 84 may be used to store information regarding, for example, patient positioning, patient movement, external forces applied to the patient, desired or preferred anatomical positions, registered patient positions, fiducial markers, frame components, sensors, and motion platform components. Stored patient position data may be devolved into data to generate a real-time automated patient positioning system in the manner generally described by examples herein.

The data storage device(s) 84 may include a random-access memory (RAM) or another type of dynamic storage device that is capable of storing updatable database information, and for separately storing instructions for execution of patient positioning by, for example, processor(s) 82. For example, a data storage device 84 may be coupled to the processor 82, and may include instructions which when executed by the processor, cause the processor to register a predetermined position of the patient based on a position of the plurality of motion platforms and the fiducial marker, actively track the location and orientation of fiducial markers attached to a patient in real-time, determine if the position of the patient has moved from the predetermined position based on the tracked position of the fiducial marker, direct the motion platform to move the patient to a preferred or ideal position, direct the motion platforms to secure the patient in the preferred anatomical position, direct dynamic stabilization to maintain patient position as external forces are applied to the patient, adjust the position of the motion platforms to realign the patient to a predetermined position when it is determined that the patient has moved from the predetermined position, and to release the patient from the preferred or ideal position for repositioning or post-instrumentation as needed. The preferred/ideal position may be a registered position of the patient, scanned image data indicating a patient position, or to a new patient position. Data storage device(s) 84 may also include a read-only memory (ROM), which may include a conventional ROM device or another type of static storage device that stores static information and instructions for processor(s) 82. Further, the data storage device(s) 84 may be integral to the controller 18, or may be provided external to, and in wired or wireless communication with the controller 18, including as cloud-based data storage components.

The data storage device(s) 84 may include non-transitory machine-readable storage medium used to store the device queue manager logic persistently. While a non-transitory machine-readable storage medium may be discussed as a single medium, the term “machine readable storage medium” should be taken to include a single medium or multiple media (e.g., a centralized or distributed database, and/or associated caches and servers) that store one or more sets of instructions. The term “machine-readable storage medium” shall also be taken to include any medium capable of storing or encoding a set of instruction for execution by the controller 18 and that causes the patient positioning system 10 to perform any one or more of the methodologies of the present invention. The term “machine-readable storage medium” shall accordingly be taken to include, but not be limited to, solid-state memories, and optical and magnetic media.

The controller 18 may include at least one data output/display device 86, which may be configured as one or more conventional mechanisms that output information to a user, including, but not limited to, a display screen on a GUI of the patient positioning system 10 or associated device with which the controller 18 may be associated. The data output/display device 86 may be used to indicate to a user a status of the patient positioning system 10 with which the controller 18 may be associated including an operation of one or more individually controlled components or subsystems associated with the patient positioning system, including but not limited to the motion platform, navigation system, sensors, compression layers, base and robot.

The controller 18 may include one or more separate external communication interfaces 88 by which the controller 18 may communicate with components that may be external to the exemplary control system such as the pressure sensor 72, navigation system 16, motion platforms 14 and robot 1000. At least one of the external communication interfaces 88 may be configured as an input port to support connecting an external CAD/CAM device storing modeling information for execution of the control functions in the patient positioning operations. Any suitable data connection to provide wired or wireless communication between the controller 18 and external and/or associated components is contemplated to be encompassed by the depicted external communication interface 88.

The controller 18 may include a patient positioning control device 90 that may be used to control movement of the motion platforms and compression layers for interfacing with a patient. The patient positioning control device 90 may operate as a part or a function of the processor 82 coupled to one or more of the data storage devices 84 and the navigation system 16, or may operate as a separate stand-alone component module or circuit in the controller 18.

All of the various components of the controller 18, as depicted in FIG. 6, may be connected internally, and to the patient positioning system 10, by one or more data/control buses 92. These data/control busses 92 may provide wired or wireless communication between the various components of the patient positioning system 10 and any associated devices, whether all of those components are housed integrally in, or are otherwise external and connected to the patient positioning system with which the controller 18 may be associated.

It should be appreciated that, although depicted in FIG. 6 as an integral unit, the various disclosed elements of the controller 18 may be arranged in any combination of subsystems as individual components or combinations of components, integral to a single unit, or external to, and in wired or wireless communication with the single unit of the exemplary control system. In other words, no specific configuration as an integral unit or as a support unit is to be implied by the depiction in FIG. 6. Further, although depicted as individual units for ease of understanding of the details provided in this disclosure regarding the controller 18, it should be understood that the described functions of any of the individually-depicted components, and particularly each of the depicted control devices, may be undertaken, for example, by one or more processors 82 connected to, and in communication with, one or more data storage device(s) 84.

The patient positioning system may operate in different modes depending on surgical requirements and procedural phase. In some cases, the motion platforms 14 may stabilize an otherwise unstable patient anatomical location (e.g., spine, bone, femur, pelvic region, torso). For example, the motion platforms 14 may be manually positioned to an initial configuration and then set in place, that is, locked via control signals from the controller to hold the actuators in place, for patient support. The initial configuration may be a preferred or ideal position for registration. The initial position may also be an unlocked configuration from which the actuators may remain active and responsive to control signals from the controller automatically or via user input for dynamic patient positioning. In some cases, after registration and locking of the motion platforms, the motion platforms actuators 36 may then be unlocked to allow repositioning. Transition between locked and unlocked modes may be controlled through the controller 18, allowing surgical personnel to select the appropriate operating mode for different phases of the procedure. The dynamic adjustment capability allows for real-time positioning and repositioning of the motion platforms to acquire and maintain preferred and ideal patient positioning proactively prior to instrumentation and reactively throughout instrumentation as conditions and undesired patient movement occur.

During operation, the patient positioning system 10 may initially function in a setup mode where surgical personnel manually position the patient on the base 12 and position the motion platforms against patient anatomical structures of interest. During this time, the controller 18 may register a predetermined or ideal position of the patient based on a position of the motion platforms and the fiducial marker. The navigation system 16 may establish baseline position references of the motion platform and patient, and the controller 18 may monitor pressure readings from the pressure sensor 72 to ensure that forces applied by the motion platform to the patient remain within acceptable ranges. To help establish position references, any number of fiducial markers 74 may be attached to the patient at the anatomical region of interest.

The navigation system 16 may begin real-time tracking of the fiducial markers for patient position monitoring. Patient position data, acquired via continuous sensor measurements of fiducial marker location and orientation, is processed by the controller. Accordingly, patient position may be known as the motion platforms 14 are manually or automatically positioned to an ideal orientation, locked in place to hold the patient in the ideal orientation, unlocked to allow further patient movement, and moved to reposition the patient as needed. It is understood that even when a patient is locked in an ideal position by the motion platforms 14, the patient may still move, and the patient's previously stable position becomes unstable due to the movement. For example, a patient's spinous process may move when the motion platforms are locked due to breathing, muscle relaxation under anesthesia, or external forces applied during surgery. In such cases, the controller in communication with the navigation system may determine if the position of the patient has moved from the predetermined position based on the tracked position of the fiducial marker, and adjust the position of the motion platforms to realign the patient to the predetermined position when it is determined that the patient has moved from the predetermined position. In other words, upon detection of undesired patient movement, the controller may calculate positioning adjustments, and coordinate movement of the motion platform to reposition the patient to the ideal position, or to another desired position.

The exemplary automated patient positioning system provides numerous benefits over current static patient positioning systems and articulated surgical tables that typically require manual adjustment and do not automatically compensate for patient movement during surgery. The automated positioning capabilities maintain consistent anatomical relationships throughout surgical procedures by continuously monitoring patient position and providing corrective adjustments without requiring manual intervention from surgical personnel. The continuous position maintenance enables robotic surgical systems to operate with greater precision by ensuring that the spatial relationships established during pre-operative planning remain consistent throughout the surgical procedure. The automated positioning system addresses detected position changes within seconds of detection, maintaining surgical workflow continuity and enabling uninterrupted progression of surgical procedures.

The reduction in positioning-related interruptions contributes to shorter overall procedure times, reducing patient exposure to anesthesia and decreasing the risk of complications associated with prolonged surgical procedures. The reduced need for manual positioning interventions enables surgical personnel to maintain focus on surgical tasks rather than positioning concerns, potentially improving surgical concentration and reducing the risk of surgical errors related to divided attention or workflow disruptions. Patient safety improvements are achieved through the integrated pressure monitoring and force feedback capabilities that prevent excessive force application during positioning adjustments and maintain patient comfort throughout surgical procedures.

It will be appreciated by those skilled in the art that changes could be made to the various aspects described above without departing from the broad inventive concept thereof. It is to be understood, therefore, that the subject application is not limited to the particular aspects disclosed, but it is intended to cover modifications within the spirit and scope of the subject application as defined by the appended claims

We claim:

1. A patient positioning system for surgical procedures comprising:
a base for supporting a patient;
a plurality of motion platforms for operatively engaging the patient;
a navigation system for tracking a position of the patient, the navigation system including a fiducial marker for attachment to the patient; and
a controller operatively in communication with the plurality of motion platforms and the navigation system.

2. The patient positioning system of claim 1, wherein each of the plurality of motion platforms is a six degrees of freedom motion platform.

3. The patient positioning system of claim 1, wherein each of the plurality of motion platforms includes a positioning arm connected to the base.

4. The patient positioning system of claim 3, wherein each of the plurality of motion platforms further includes a static base connected to the positioning arm, a dynamic platform for operatively engaging the patient, and a plurality of actuators connecting the static base to the dynamic platform for movement of the dynamic platform in three-dimensional space relative to the static base.

5. The patient positioning system of claim 1, wherein the plurality of motion platforms includes at least two motion platforms positioned about each lateral side of the base.

6. The patient positioning system of claim 1, further comprising a compression layer extending from at least one of the plurality of motion platforms for interfacing with the patient.

7. The patient positioning system of claim 6, wherein the compression layer is a beanbag layer.

8. The patient positioning system of claim 7, wherein the beanbag layer includes a vacuum-actuated beanbag positioner.

9. The patient positioning system of claim 1, wherein the navigation system is an optical or electromagnetic navigation system.

10. The patient positioning system of claim 1, further comprising a sensor operatively in communication with the controller for detecting an attribute of the patient.

11. The patient positioning system of claim 10, wherein the sensor is a pressure sensor.

12. The patient positioning system of claim 1, wherein the controller includes a memory having computer instructions executable to:
register a predetermined position of the patient based on a position of the plurality of motion platforms and the fiducial marker;
track the position of the fiducial marker in real time via the navigation system;
determine if the position of the patient has moved from the predetermined position based on the tracked position of the fiducial marker; and
adjust the position of at least one of the plurality of motion platforms to realign the patient to the predetermined position when it is determined that the patient has moved from the predetermined position.

13. The patient positioning system of claim 1, wherein the navigation system includes a navigation sensor for detecting a position of the fiducial marker.

14. A patient positioning system for surgical procedures, comprising:
a base for supporting a patient;
a plurality of motion platforms for operatively engaging the patient, wherein each of the plurality of motion platforms is a six degrees of freedom motion platform;
a navigation system for tracking a position of the patient, the navigation system including a fiducial marker for attachment to the patient; and
a controller operatively in communication with the plurality of motion platforms and the navigation system, wherein the controller includes a memory having computer instructions executable to:
register a predetermined position of the patient based on a position of the plurality of motion platforms and the fiducial marker,
track the position of the fiducial marker in real time via the navigation system,
determine if the position of the patient has moved from the predetermined position based on the tracked position of the fiducial marker, and
adjust the position of at least one of the plurality of motion platforms to realign the patient to the predetermined position when it is determined that the patient has moved from the predetermined position.

15. The patient positioning system of claim 14, further comprising a pressure sensor operatively in communication with the controller for detecting applied forces on the patient and providing feedback to the controller for detecting patient movement.

16. The patient positioning system of claim 14, further comprising a compression layer extending from at least one of the plurality of motion platforms for interfacing with the patient.

17. The patient positioning system of claim 16, wherein the compression layer is a beanbag layer.

18. The patient positioning system of claim 17, wherein the beanbag layer includes a vacuum-actuated beanbag positioner.

19. The patient positioning system of claim 14, wherein the navigation system is an optical or electromagnetic navigation system.
